(19) United States
(12) Patent Application Publication
(10) Pub. No.: US 2025/0302655 A1
(43) Pub. Date: Oct. 2, 2025

(54) LATERAL ADJUSTABLE CERVICAL COLLAR POSTERIOR PANEL

(71) Applicant: Aspen Medical Products, LLC, Irvine, CA (US)

(72) Inventors: Jane Price, Anaheim, CA (US); Erik Zimmer, Oceanside, CA (US)

(21) Appl. No.: 19/094,754

(22) Filed: Mar. 28, 2025

Related U.S. Application Data
(60) Provisional application No. 63/572,687, filed on Apr. 1, 2024.

(51) Int. Cl. A61F 5/055

(52) U.S. Cl. CPC A61F 5/055

(57) ABSTRACT

A back posterior collar assembly for a cervical collar is described. The back posterior collar assembly features one or more cervical panels and an attachment panel. The attachment panel includes a central support member, a first extension member, and a second extension member. The central support member is coupled to the one or more cervical panels. The first extension member is coupled to the central support member and configured to be extended laterally toward and away from the central support member. The second extension member is coupled to the central support member and configured to be extended laterally toward and away from the central support member in the opposite direction.

### CROSS-REFERENCE TO RELATED APPLICATIONS

This application claims the benefit of priority on U.S. Provisional Application No. 63/572,687 filed Apr. 1, 2024, the entire contents of which are incorporated by reference herein.

### FIELD

Embodiments of the disclosure relate to the field of orthopedic braces. More specifically, one embodiment of the disclosure relates to an adjustable posterior panel configured for attachment to a cervical collar.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Cervical collars are used to substantially immobilize the cervical spine for treatment of a neck or cervical spine condition. These collars may assist in the treatment of whiplash, back surgery, and other similar conditions, where support for the head and neck of a wearer (hereinafter, “patient”) is needed. For some orthopedic braces, the cervical collar is merely one component of the brace, which is configured to prevent movement in a wearer's spine from the cervical region (neck and upper back region).

A cervical collar includes an anterior collar assembly and a posterior collar assembly. Currently, the posterior collar assembly includes a posterior panel, which is attached to the anterior collar assembly by coupling nylon straps extending from the posterior panel to corresponding fasteners of the anterior collar assembly. Currently, the posterior panel is a single-piece or multi-piece component, where the coupling to the anterior collar assembly of the cervical collar may be achieved only through a single, unique deployment scheme. The conventional posterior panels offer no lateral adjustment to account for the neck size of the patient as these posterior panel have a predefined length. As a result, conventional posterior cervical collar panels, in some cases, fail to provide a suitable fit in which straps of the posterior cervical collar panel are inappropriately attached to complementary fasteners situated on lateral sides of a front collar body.

More specifically, for patients with small neck circumferences, the straps associated with a singular-sized, conventional posterior cervical collar panel tend to either (i) partially extend beyond the complementary fasteners to impede the patience's peripheral sight or (ii) excess portions of the strap sag adjacent to the lateral sides of the front collar body. In contrast, for patients with substantially large neck circumferences, the straps may barely attach to the complementary fasteners, so that the cervical collar may be uncomfortable to the patient and fail to provide sufficient stabilization of the patient's neck.

Conventional, cervical collar back panel architectures need improvement to mitigate the donning difficulties and develop a back panel architecture that finds a balance between immobilization of the cervical spine and patient comfort to ensure compliance with wearing instructions.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 is a perspective view of an exemplary embodiment of a cervical collar including a posterior collar assembly.

FIG. 2 is an illustrative embodiment of the posterior collar assembly of FIG. 1, which includes a cervical collar posterior panel and a laterally adjustable attachment panel.

FIG. 3A is a posterior view of a first exemplary embodiment of the posterior collar assembly is shown, including the cervical collar posterior panel attached to the attachment panel.

FIG. 3B is an illustrative embodiment of the attachment panel of FIG. 3A, where the attachment panel includes a central support member, a first laterally adjustable extension member, and a second laterally adjustable extension member.

FIG. 4 is a perspective view of an illustrative embodiment of the posterior collar assembly of FIG. 3A including fastening straps for secured coupling of the first laterally adjustable extension member and the second laterally adjustable extension member of FIGS. 3A-3B.

FIG. 5A is a posterior view of the second laterally adjustable extension member of the attachment panel illustrated in FIG. 4, where the second laterally adjustable extension member is positioned into a fully extended position.

FIG. 5B is a posterior view of the second laterally adjustable extension member of the attachment panel illustrated in FIG. 5A, where the second laterally adjustable extension member is placed into an extended state and a first end of a connection strap is attached to a fastener positioned on the second laterally adjustable extension member.

FIG. 5C is a posterior view of the second laterally adjustable extension member of the attachment panel illustrated in FIG. 5B. is positioned in a shortened position.

FIG. 6A is a posterior view of a second exemplary embodiment of the attachment panel.

FIG. 6B is a perspective view of the attachment panel of FIG. 6A.

FIG. 7A is a side view of the second laterally adjustable extension member of FIG. 6A-6B, where the guide fastener is placed in an opened state.

FIG. 7B is a side view of the first laterally adjustable extension member of FIG. 7A, where a lever of the guide fastener situated on the second laterally adjustable extension member of FIG. 7A is placed in a closed state.

FIG. 8A is an illustrative embodiment of a perspective, posterior view of a portion of the attachment panel of FIG. 6A.

FIG. 8B is an illustrative embodiment of a perspective side view of the second laterally adjustable extension member of the attachment panel of FIG. 8A.

### DETAILED DESCRIPTION

Embodiments of the present disclosure generally relate to a posterior collar assembly that features a cervical collar posterior panel, which is the rear component for a cervical collar. For these embodiments, cervical collar posterior panel features one or more cervical panel members coupled to an attachment panel, albeit the cervical collar posterior panel may be constructed as a single integrated component. Herein, for this embodiment, a pair of cervical panel members are designed in which one of the cervical panel members is positioned to support the occipital region (e.g., occipital bone) of the patient's skull while the other cervical panel member is positioned to receive support by the upper trapezius region (e.g., upper trapezius muscle area proximate to the cervical spine) of the patient. Depending on its positioning to a central member of the attachment panel, when worn, a cervical panel member may feature (i) a concave shape to rest against and support the occipital region of the patient or (ii) a convex shape to rest against and be supported by the upper trapezius region of the patient.

According to these embodiment of the disclosure, the attachment panel features a central support member, a first laterally adjustable extension member, and a second laterally adjustable extension member. The central support member features a centralized aperture with periphery members surrounding the aperture. From a posterior perspective, the top/bottom periphery members are configured with a gradual concave form. In particular, a first (top) periphery member is configured with an incline angle (e.g., upward slope) tending to veer away from an anterior side of the cervical collar posterior panel while a second (bottom) periphery member is configured with a decline angle veering from an anterior side of the cervical collar posterior panel. These concave-shaped top/bottom periphery members assist in the orientation of the cervical panels to support the occipital region of the patient and be supported by the upper trapezius region of the patient.

For a first embodiment of the disclosure, the laterally adjustable extension members extend in opposite directions from the central support member and are constructed to provide lateral support for straps that are attached to a front collar body of the cervical collar. Each of the laterally adjustable extension members includes a first extension panel and a second extension panel, which is moveably coupled to the first extension panel. The first extension panel includes one or more guides (e.g., guide channels), which enable the second extension panel to move laterally (slide) close to or away from the central support member.

More specifically, the first extension panel may include one or more slots operating as guides. For this embodiment, the second extension panel may include a guide fasteners inserted through the slots and secured so that the second extension panel is moveably coupled to the first extension panel. This enables the second extension panel to be adjusted in connection with the first extension panel to increase the overall length of one or more of the laterally adjustable extension members in order to accommodate for a large neck circumferences and to decrease the overall length of one or more of the laterally adjustable extension members to account for small neck circumferences. The degree of laterally adjustable length change provided by the laterally adjustable extension members may range up to two additional extension panel lengths. As a result, the posterior collar assembly may account for different patient neck (or head) sizes for a more comfortable fit of the cervical collar to increase wearing compliance.

For to a second embodiment of the disclosure, each of the laterally adjustable extension members includes a first extension panel and a second extension panel. However, second extension panel includes a fastener (e.g., cam latch) that, when placed into an opened state, allows the second extension panel to move laterally close to or away from the central support member. However, when the fastener is placed into a closed state, a downward pressure is applied so that the second extension panel is precluded from movement from the first extension panel.

### I. Terminology

In the following description, certain terminology is used to describe aspects of the invention. For example, each of the terms “member” and “panel” may be construed as a physical component, where the physical component may be part of an orthosis such as a cervical collar for example. In certain situations, the member (or panel) may rely on one or more fasteners (hereinafter, “fastener(s)”) for attachment of that member (or panel) to a different member (or panel) that features complementary fastener(s). For example, a first member may include a “loop” type fastener to which a “hook” type fastener located on a second member may be attached or the first member may include a hook-type fastener for attachment to a loop-type fastener located on the second member.

The terms “attach” and “connect” as well as other verb tenses of these terms (e.g., attached, attaching, connected, connecting, etc.) may be construed as physically coupling two items together. This physical coupling may be a direct coupling of the two items using fastener(s) or an indirect coupling that relies on one or more intermediary members to which the two items are connected. The term “fastener” may be construed as any physical component used to attach different items together. An illustrative example of different types of fasteners and fastening techniques may include, but are not limited or restricted to a snap, button, clasp, clip, hook, or loop element (e.g., Velcro®), buckle, dowel/cap, rivet, screw, bolt/nut, adhesive, heat seal (or melting), or other component that is connected to its complementary fastener.

The term “concave” may be construed as a curvature in a first direction (e.g., upward like an interior of a circle) while the term “convex” may be construed as a curvature in a second direction (e.g., downward like an exterior of a circle”). It is contemplated that these curvatures may be alterable with a change in viewpoint, where a surface may be convex from a posterior viewpoint and concave from an anterior viewpoint. As a result, for the subject application, the viewpoints for the figures are identified.

The term “rigid” with respect to a member or portion of a member may be construed as the member could be permanently deformed or broken if bent or twisted by at least 90°.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As this invention is susceptible to embodiments of many different forms, it is intended that the present disclosure is to be considered as an example of the principles of the invention and not intended to limit the invention to the specific embodiments shown and described.

### II. General Architecture

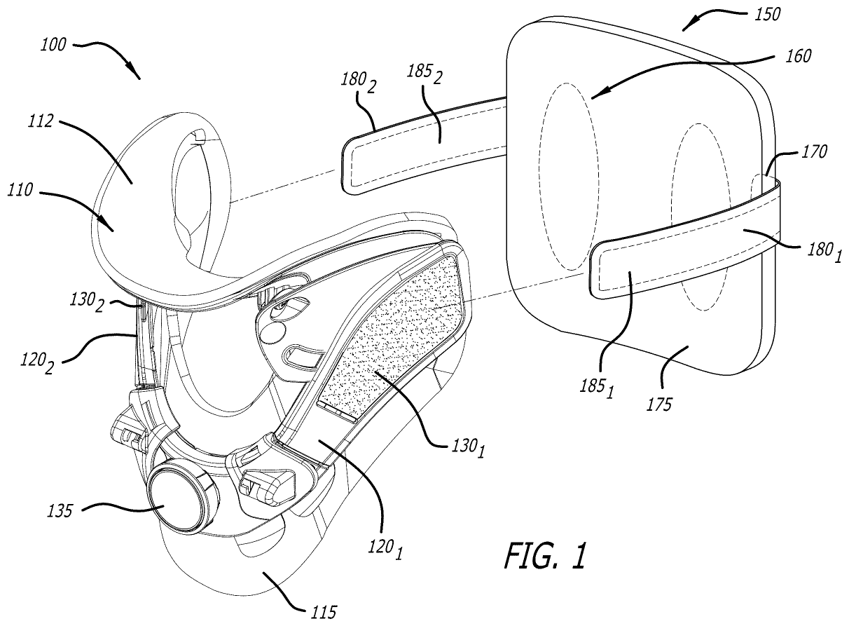

Referring to FIG. 1, a perspective view of an exemplary embodiment of a cervical collar 100, which includes an anterior collar assembly 110 and a posterior collar assembly 150, is shown, According to one embodiment, the anterior collar assembly 110 features a chin piece 112, torso rest 115, first and second side members 120₁-120₂, a first fastener 130₁ positioned over the first side member 120₁, a second fastener 130₂ positioned over the second side member 120₂, and a height control knob 135 that, when rotated, causes adjustment in the positioning of the chin piece 112. Of course, other adjustable collar assemblies or even non-adjustable collar assemblies may be used in combination with the posterior collar assembly 150.

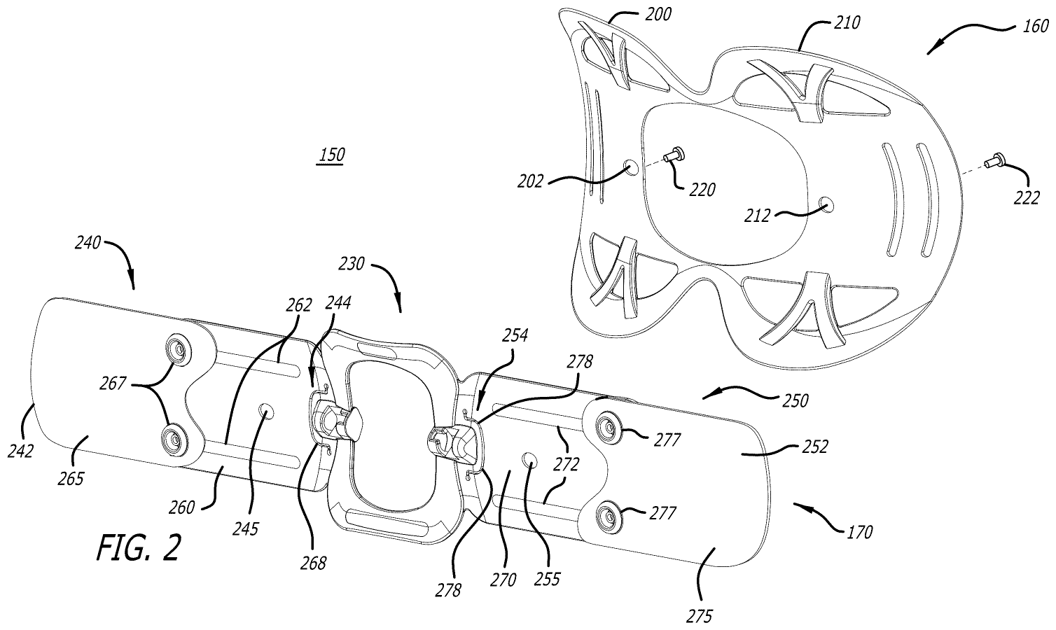

As shown in FIG. 1, the posterior collar assembly 150 features a cervical collar posterior panel 160, which is coupled to an attachment panel 170 and to a pad 175 that is in contact with the neck/head area of the patient when the cervical collar 100 is worn. The attachment panel 170 includes straps 180₁-180₂, which are coupled to the attachment panel 170. An end of a first strap 180₁ includes a fastener 185₁ complementary to the first fastener 130₁ while an end of a second strap 180₂ includes a fastener 185₂ complementary to the second fastener 130₂. The straps 180₁-180₂ are laterally adjustable based on the lateral adjustability of extension panels forming the attachment panel 170 as shown in FIG. 2.

Referring now to FIG. 2, an illustrative embodiment of the posterior collar assembly 150 of FIG. 1 is shown. The posterior collar assembly 150 includes the cervical collar posterior panel 160 and the laterally adjustable attachment panel 170. The cervical collar posterior panel 160 features a first cervical panel 200 and a second cervical panel 210. Both the cervical panels 200 and 210 include apertures 202 and 212 to align with fastener receptacle 245 and 255_{\} adapted to receive fasteners 220 and 222 inserted through the apertures 202 and 212, respectively.

The attachment panel 170 includes a central support member 230, a first extension member 240, and a second extension member 250. The central support member 230 is configured to retain the first cervical panel 200 and the second cervical panel 210, which are attached together through one or more fasteners (not shown). Alternatively, the posterior collar assembly 150, inclusive of the cervical collar posterior panel 160 and the attachment panel 170, may be constructed as a single integrated component.

The first extension member 240 includes a distal region 242 and a proximal region 244. The proximal region 244 is coupled to the central support member 230 while the distal region 242 is laterally adjusted (e.g., increase or decrease length of the first extension member 240) so that distal region 242 may contact the first side member 120₁ when the cervical collar 100 is worn. More specifically, the first extension member 240 includes a first extension panel 260 and a second extension panel 265, where the first extension panel 260 is coupled to the central support member 230 and the second extension panel 265 is moveably coupled to the first extension panel 260.

The first extension panel 260 includes one or more guides 262 in which a corresponding guide fasteners 267 are locked therein to enable the second extension panel 265 to move laterally towards or away from the central support member 230. As shown, the guide(s) 262 may correspond to one or more slots. The guide fastener 267 may include a female fastener adapted to receive a male fastener (e.g., a rivet) protruding through the slot(s) 262 for attachment. This lateral adjustment allows the first extension member 240 to increase in length by approximately the length of the guide(s) 262. For example, where the guide(s) 262 are channel(s) that are 2-3 inches in length, the first extension member 240 may be configured to increase in size by approximately three inches in length.

Similarly, the second extension member 250 includes a distal region 252 and a proximal region 254. The proximal region 254 is coupled to the central support member 230 while the distal region 252, when laterally adjusted, may contact the second side member 120₂ when the cervical collar 100 is worn. More specifically, the second extension member 250 includes a third extension panel 270 and a fourth extension panel 275, where the third extension panel 270 is coupled to the central support member 230 and the fourth extension panel 275 is moveably coupled to the third extension panel 270. The third extension panel 270 includes one or more guides 272 in which guide fasteners 277 are locked thereto to enable the fourth extension panel 275 to move laterally towards or away from the central support member 230 to extend the length of the second extension member 250.

Both the first extension panel 260 and the third extension panel 270 feature a respective slot 268 and 278, each is configured to receive a strap (not shown) that is used for connectivity to the anterior collar assembly 110 of FIG. 1.

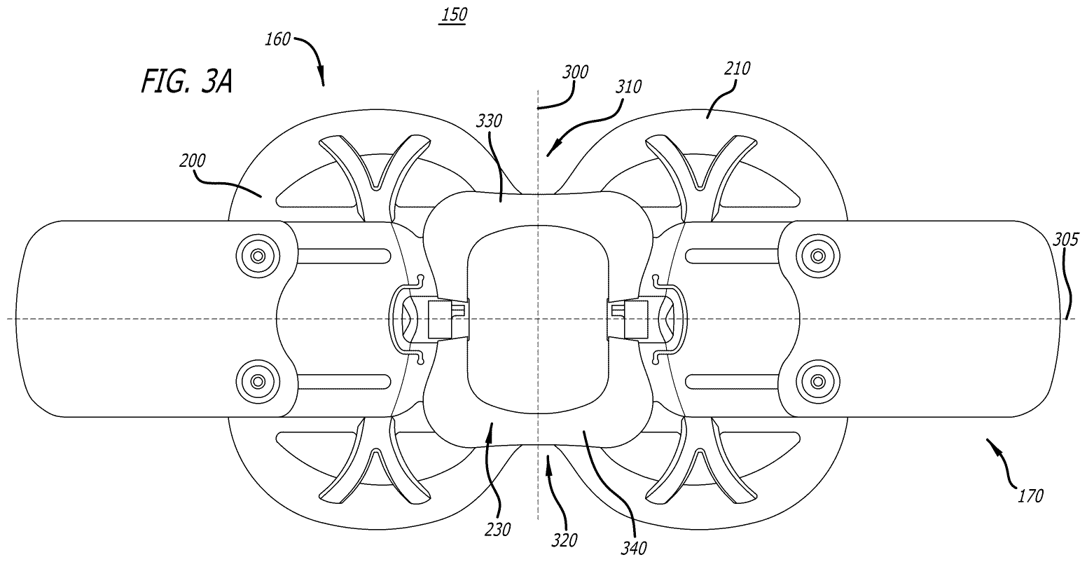

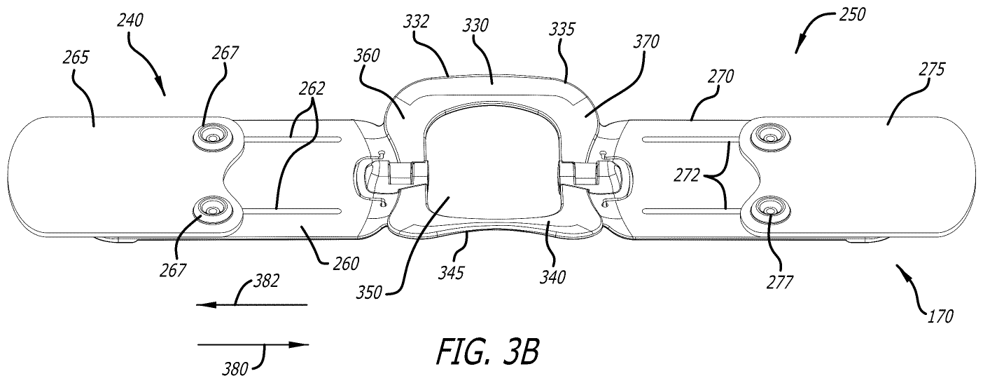

Referring now to FIG. 3A, a posterior view of a first exemplary embodiment of the posterior collar assembly 150, including the cervical collar posterior panel 160 attached to the attachment panel 170, is shown. The cervical collar posterior panel 160 further includes the first cervical panel 200 and the second cervical panel 210, which are positioned symmetric to each other from a traverse axis 300 intersecting the central support member 230. Additionally, the first cervical panel 200 and the second cervical panel 210 and/or the first extension member 240 and the second extension member 250 are symmetric to each other in accordance with a longitudinal axis 305.

Intersection regions 310 and 320 of the first and second cervical panels 200 and 210 feature a curved structure that is complementary to the curved form of the anterior surfaces of a first periphery member 330 and a second periphery member 340 of the central support member 230. As shown in FIGS. 3A-3B, for a posterior viewpoint, the first cervical panel 200 features a convex configuration and is positioned at a lateral offset distance from an anterior surface 335 of the first periphery member 330. Also, the second cervical panel 210 features a convex configuration and is positioned at a lateral offset distance from an anterior surface 345 of the second periphery member 340.

Referring still to FIGS. 3A-3B, the first cervical panel 200 is configured and shaped to conform to the occipital region of the patient. The first cervical panel 200 is positioned at an offset distance from the anterior surface 335 of the first periphery member 330 to allow for articulation at an attachment point to the central support member 230. Similarly, the second cervical panel 210 features a concave configuration, is positioned at an offset distance from the anterior surface 345 of the second periphery member 340 with articulation at the attachment point. When the cervical collar is worn, the first and second cervical panels 200 and 210 are interchangeable as both of these cervical panels 200 and 210 may be used to support the occipital region or the upper trapezius region of the patient.

As shown in detail in FIG. 3B, an illustrative embodiment of the attachment panel 170 of FIG. 1 is shown, where the attachment panel 170 includes the central support member 230, the first extension member 240 and the second extension member 250, where the first and second extension members 240 and 250 are laterally adjustable. According to this embodiment, the central support member 230 features a centralized aperture 350 formed by a plurality of periphery members, including the first periphery member 330 and the second periphery member 340, as well as a third periphery member 360 and a fourth periphery member 370. Herein, from a posterior viewpoint, the first periphery member 330 is configured with a concave (e.g., upward sloping) surface from the aperture 350 to its edge 332 while the edge 332 is convex in form between distal ends of the first periphery member 330. Similarly, the second periphery member 340 is configured with a concave surface from the aperture 350 to its edge 342 and convex in form from its distal ends.

Referring still to FIG. 3B, the first extension member 240 includes the first extension panel 260 with the plurality of guides 262 to allow the second extension panel 265, as governed by a corresponding plurality of guide fasteners 267, to move laterally as designated by arrows 380 and 382. According to one embodiment of the disclosure, the first extension panel 260 includes one or more guides 262 in which a corresponding guide fasteners 267 (e.g., female fastener(s) adapted to receive a male fastener protruding through the slot(s) 262) may be locked in place to enable the second extension panel 265 to move laterally towards or away from the central support member 230 as represented by arrows 380 and 382.

According to another embodiment of the disclosure, the diameter of the guide fasteners 267 is slightly smaller than a width of the guides 262 so that frictional resistance is applied from the edges of the guides 262 to the guide fasteners 267 of the second extension panel 265. This sizing is designed to prevent unencumbered lateral movement of the second extension panel 265 in connection with the first extension panel 260. However, according to yet another embodiment of the disclosure, the diameter of the guide fasteners 267 is smaller than the width of the guides 262 so that no friction resistance is applied by the edges of the guides 262 to the guide fasteners 267 of the second extension panel 265. Hence, the second extension panel 265 experiences free, unencumbered lateral movement and is secured through use of a fastening strap 180₁ (see FIGS. 1 & 4) inserted through the slot 268 for attachment to the first fastener 130₁ of the anterior collar assembly 110 of FIG. 1. For both of these deployments, a fastening strap may be used to further secure the neighboring extension panels together.

Similarly, the second extension member 250 includes the third extension panel 270 and the fourth extension panel 275, where the third extension panel 270 is coupled to the central support member 230 and the fourth extension panel 275 is moveably coupled to the third extension panel 270 in a similar manner as described above. The third extension panel 270 includes one or more guides 272 in which a portion of the guide fasteners 277 are configured to be inserted through the guide(s) 272 and secure the third extension panel 270 to the fourth extension panel 275. As a result, the fourth extension panel 275 is adapted to move laterally towards or away from the central support member 230 to extend the length of the second extension member 250.

In summary, with respect to the configuration of the first and second extension members 240 and 250, the attachment panel 170 is able to be laterally re-sized to accommodate patients with smaller or larger neck circumferences to provide a more comfortable fit to wear the cervical collar. Additionally, the re-sizing of the extension members 240 and 250 provide a universal product that can accommodate a larger size range instead of requiring two or more different sized products to accommodate the same range.

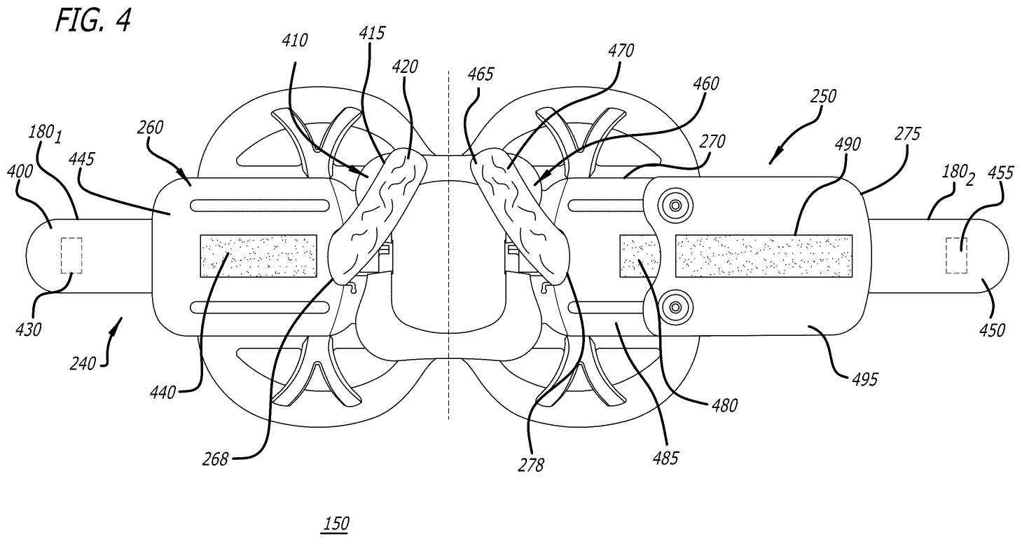

Referring now to FIG. 4, a perspective view of an illustrative embodiment of the posterior collar assembly 150 of FIG. 3A is shown, inclusive of fastening straps 180₁-180₂. The first fastening strap 180₁ includes a first end 400, which features a fastener 430 (e.g., any type of fastening mechanism including a rivet, screw, adhesive material, male/female snap combination, Velcro® or other loop or hook material, etc.) for connecting the attachment panel 170 to the anterior collar assembly 110. For example, the fastener 430 is configured to connect the attachment panel 170 to the first fastener 130₁ (positioned on the first side member 120₁ of the anterior collar assembly 110 as shown in FIG. 1).

According to this embodiment of the disclosure, a first positioning strap 410 includes a first end coupled to an anterior side of the first extension panel 260 by a fastener (not shown). As identified above, the fastener may include any type of fastening component or material such as a rivet, screw, adhesive material, snap, hook/loop, or the like. The first positioning strap 410 is inserted through slot 268 formed in the first extension panel 260, where a second end of the first positioning strap 410 includes a fastener 420 positioned on a first side 415 of the first positioning strap 410.

As shown, after insertion through the slot 268, the fastener 420 comes into contact with and secures itself to a complementary fastener 440. The complementary fastener 440 is positioned on a posterior surface 445 of the first extension panel 260. Although not shown, the second extension panel 265 is positioned posteriorly on the first extensional panel 260 and may feature a fastener positioned on a posterior surface of the second extension panel 265, similar to the configuration as shown and described below for the second extension member 250. Therefore, after lateral adjustment of the second extension panel 265 in relation to the central support member 230, the fastener 420 would be attached to complementary fasteners on the first/second extension panels 260/265.

Additionally, the second fastening strap 180₂ includes a first end 450, which features a fastener 455 (e.g., any type of fastening mechanism including a rivet, screw, adhesive material, male/female snap combination, Velcro® or other loop or hook material, etc.) for connecting the attachment panel 170 to the anterior collar assembly 110. For example, the fastener 455 is configured to connect the attachment panel 170 to the second fastener 130₂ (positioned on the second side member 120₂ of the anterior collar assembly 110 as shown in FIG. 1).

According to this embodiment of the disclosure, a second positioning strap 460 includes a first end coupled to an anterior side of the third extension panel 270 by a fastener (not shown). The second positioning strap 460 is inserted through slot 278 formed in the third extension panel 270, where a second end of the second positioning strap 460 includes a fastener 470 positioned on a first side 465 of the second positioning strap 460.

Similarly to the first positioning strap 410, after insertion through the slot 278 and lateral adjustment of the fourth extension panel 275 in relation to the central support member 230, the fastener 470 comes into contact with and secures itself to a complementary fastener 480 positioned on a posterior surface 445 of the second extension panel 275. Additionally, the fastener 470 may be placed into contact with and secures to fastener 490, which is positioned on a posterior surface 495 of the fourth extension panel 275 to further secure the extended length of the second extension member 250, where the fasteners 480 and 490 are aligned with each other.

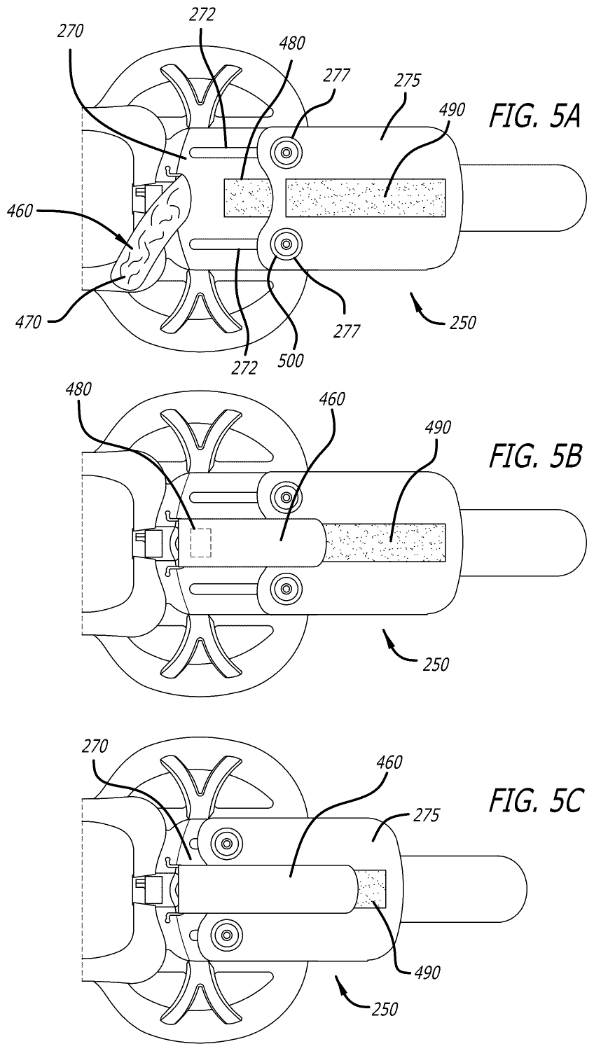

Referring to FIG. 5A, a posterior view of the second laterally adjustable extension member 250 of the attachment panel 170 illustrated in FIG. 4, where the second laterally adjustable extension member 250 is positioned into a fully extended state. Herein, the fourth extension panel 275 is laterally adjusted so that the guide fasteners 277 extend to distal ends 500 of the guides 272. Thereafter, as shown in FIG. 5B, the fastener 470 of the second positioning strap 460 engages with the fasteners 480 and 490 of the third and fourth extension panels 270 and 275, respectively.

Referring now to FIG. 5C, a posterior view of the second laterally adjustable extension member 250 of the attachment panel 170 is shown. Herein, the second laterally adjustable extension member 250 is positioned into a non-extended state with the fourth extension panel 275 substantially overlay the third extension panel 270. Hence, the second positioning strap 460 is coupled to the fastener 490; however, the fastener 480 is covered by fourth extension panel 275 and is not exposed for use by the second positioning strap 460.

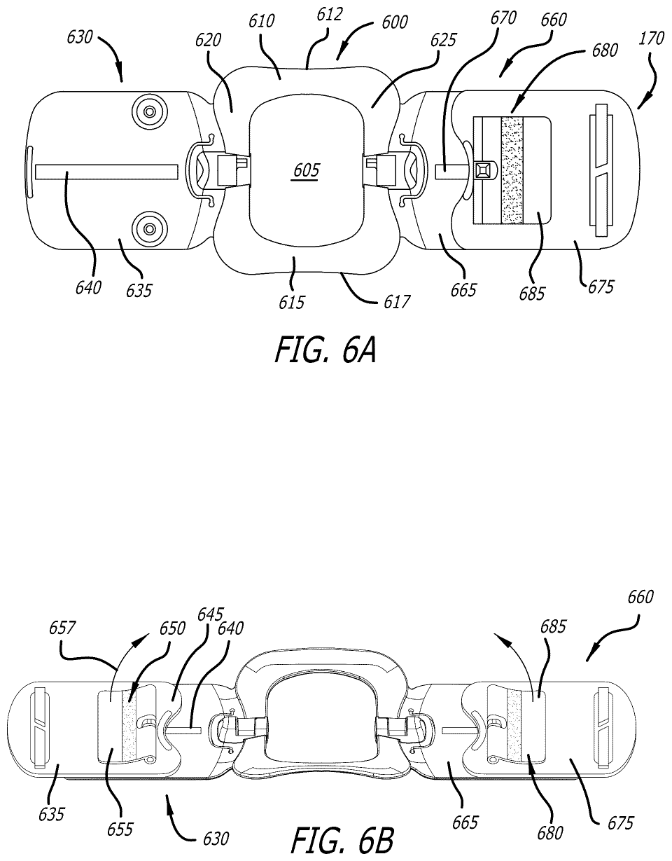

Referring now to FIG. 6A, a posterior view of a second exemplary embodiment of the attachment panel 170, including a central support member 600, a first (laterally adjustable) extension member 630 and a second (laterally adjustable) extension member 660, is shown. According to this embodiment, the central support member 600 features an architecture identical to the central support member 230 of FIG. 2, where the central support member 600 features a centralized aperture 605 formed by a plurality of periphery members, including a first periphery member 610, a second periphery member 615, a third periphery member 620, and a fourth periphery member 625. As shown in FIGS. 6A-6B, from a posterior viewpoint, the first periphery member 610 is configured with a concave (e.g., upward sloping) surface from the aperture 605 to its edge 612 while the edge 612 is convex in form between distal ends of the first periphery member 610. Similarly, the second periphery member 615 is configured with a concave surface from the aperture 605 to its edge 617 and convex in form from its distal ends.

Referring still to FIGS. 6A-6B, the first extension member 630 includes the first extension panel 635 with one or more guides 640 to allow a second extension panel 645 to move laterally. According to this embodiment of the disclosure, the first extension panel 635 includes a single guide 640 in which a corresponding guide fastener 650 (e.g., cam latch) is coupled. When a lever 655 of the guide fastener 650 is positioned downward, as shown, the second extension panel 645 is pressed against the first extension panel 635, and as the result, the first extension member 630 is placed into a locked state. However, when the lever 655 of the guide fastener 650 is rotated upward and placed into an unlocked state (as represented by arrow 657), the second extension panel 645 is released and able to move laterally towards or away from the central support member 600.

Similar in architecture to the first extension member 630, the second extension member 660 includes a third extension panel 665 and a fourth extension panel 675, where the third extension panel 665 is coupled to the central support member 600 and the fourth extension panel 675 is moveably coupled to the third extension panel 665. The third extension panel 665 includes one or more guides 670 in which a portion of a guide fastener 680 of the fourth extension panel 675 (e.g., cam latch) is configured to be inserted through the guide 670 and secured thereto. When the guide fastener 680 is placed into a locked state, a pressure is applied to secure the third extension panel 665 to the fourth extension panel 675. When placed into an unlocked state, by rotating a lever 685 of the guide fastener 680 in an upward direction, the fourth extension panel 675 is adapted to move laterally towards or away from the central support member 600 to extend the length of the second extension member 660.

In summary, with respect to the configuration of the first and second extension members 630 and 660, the attachment panel 170 is able to be laterally adjusted in sizing to accommodate patients with smaller or larger neck circumferences to provide a more comfortable fit to wear the cervical collar and/or provide the cervical product that can accommodate a larger size range than conventional cervical products.

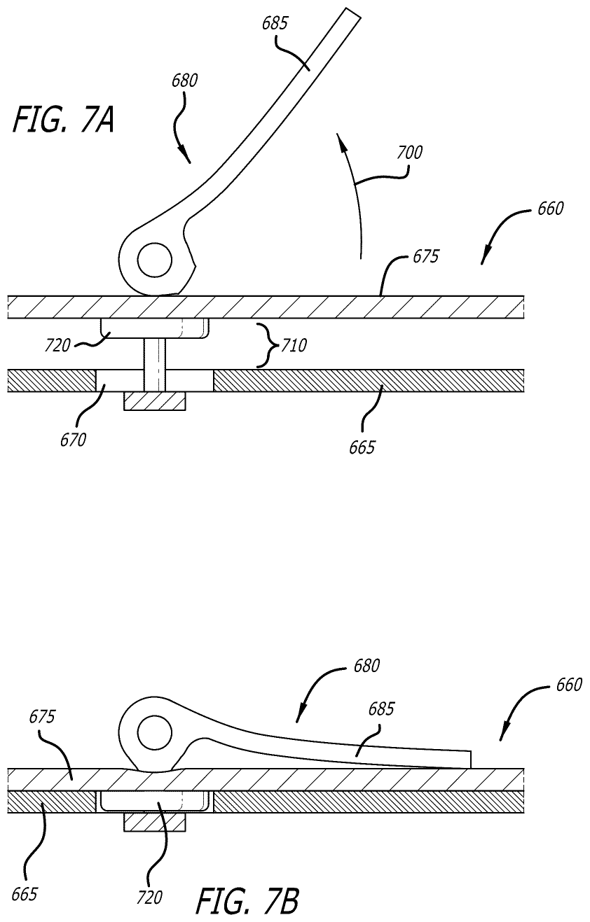

Referring to FIG. 7A, a side view of the second laterally adjustable extension member 660 of FIG. 6A-6B, where the guide fastener 680 is placed in an opened state. Herein, the lever 685 is rotated upward as represented by arrow 700 to create a separation distance 710 greater than a few millimeters between the third extension panel 665 and the fourth extension panel 675. This separation distance 710 is created by upward movement of an insert 720 of the guide fastener 680, and thus, the fourth extension panel 675 is able to laterally move in relation to the third extension panel 665 as defined by the guide 670.

As shown in FIG. 7B, the lever 685 of the guide fastener 680 situated on the second laterally adjustable extension member 660 of FIG. 7A is placed in a closed state to prevent lateral movement of the fourth extension panel 675 in relation to the third extension panel 665. Herein, the insert 720 is moved downward so that the third extension panel 665 now applies pressure against the fourth extension panel 675 and prevents lateral movement of the fourth extension panel 675.

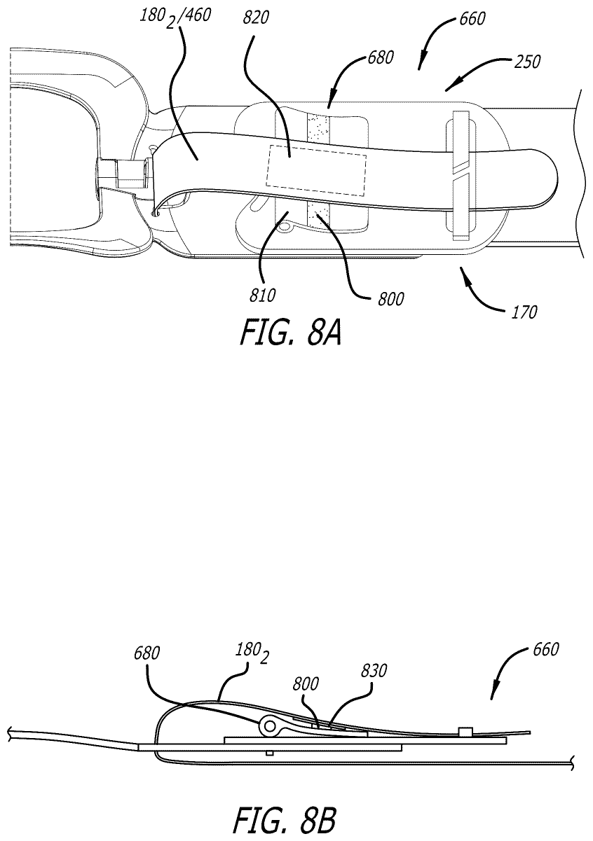

Referring now to FIG. 8A, an illustrative embodiment of a perspective, posterior view of a portion of the attachment panel 170 (second extension member 250) of FIG. 6A is shown, where the fastening strap 180₂ (or positioning strap 460 of FIG. 4) is attached to the guide fastener 680 of the second laterally adjustable extension member 660. Herein, the guide fastener 680 features fastener 800, which is positioned on a top surface 810 of the guide fastener 680. For example, the fastener 800 may be a hook or loop fastener, in which a bottom surface 820 of the fastening strap 180₂ includes a complementary fastener 830 to the fastener 800 (e.g., loop or hook fastener). The fastener 800 mitigates movement of the fastening strap 180₂ (or positioning strap 460) after tightening. For instance, the operability of the fastener 800 is shown in connection with the fastening strap 180₂, which is represented by a perspective side view of the second laterally adjustable extension member 660 shown in FIG. 8B. As alluded to above, in lieu of the fastening strap 180₂, the second positioning strap 460 of FIG. 4 may be utilized in combination with the guide fastener 680. The same construction would be conducted for the first extension member 240.

In the foregoing description, the invention is described with reference to specific exemplary embodiments thereof. However, it will be evident that various modifications and changes may be made thereto without departing from the broader spirit and scope of the invention as set forth in the appended claims.

What is claimed is:

1. A posterior collar assembly for a cervical collar, comprising:
one or more cervical panels; and
an attachment panel including (i) a central support member coupled to the one or more cervical panels, (ii) a first extension member coupled to the central support member and configured to be extended laterally toward and away from the central support member, and (iii) a second extension member coupled to the central support member and configured to be extended laterally toward and away from the central support member.

2. The posterior collar assembly of claim 1, wherein the first extension member is laterally adjustable in a first direction away from the central support member and the second extension member is laterally adjustable in a second direction away from the central support member.

3. The posterior collar assembly of claim 1, wherein the first extension member comprises a first extension panel and a second extension panel, the first extension panel being coupled to the central support member and the second extension panel is moveably coupled to the first extension panel to laterally move in a first direction away from the central support member and in a second direction toward the central support member.

4. The posterior collar assembly of claim 3, wherein the second extension member comprises a third extension panel and a fourth extension panel, the third extension panel being coupled to the central support member and the fourth extension panel is moveably coupled to the third extension panel to laterally move in the second direction away from the central support member and in the first direction toward the central support member.

5. The posterior collar assembly of claim 3, wherein the first extension panel includes one or more guides and the second extension panel includes one or more guide fasteners slidably coupled to the one or more guides.

6. The posterior collar assembly of claim 3, wherein the second extension panel includes a guide fastener operating as a cam latch that, when a lever of the guide fastener is depressed towards the first extension panel, the second extension panel is secured to avoid movement of the second extension panel in relation to the first extension panel.

7. The posterior collar assembly of claim 3, wherein the first extension member and the second extension member are symmetrical and located on opposite lateral sides of the central support member.

8. The posterior collar assembly of claim 1 being coupled to an anterior collar assembly to form a cervical collar.

9. An attachment panel for a cervical collar, comprising:
a central support member configured for coupling to one or more cervical panels of the cervical collar;
a first extension member coupled to the central support member and configured to be extended laterally toward and away from the central support member; and
a second extension member coupled to the central support member and configured to be extended laterally toward and away from the central support member.

10. The attachment panel of claim 9, wherein the first extension member is laterally adjustable in a first direction away from the central support member and the second extension member is laterally adjustable in a second direction away from the central support member.

11. The attachment panel of claim 9, wherein the first extension member comprises a first extension panel and a second extension panel, the first extension panel being coupled to the central support member and the second extension panel is moveably coupled to the first extension panel to laterally move in a first direction away from the central support member and in a second direction toward the central support member.

12. The attachment panel of claim 11, wherein the second extension member comprises a third extension panel and a fourth extension panel, the third extension panel being coupled to the central support member and the fourth extension panel is moveably coupled to the third extension panel to laterally move in the second direction away from the central support member and in the first direction toward the central support member.

13. The attachment panel of claim 11, wherein the first extension panel includes one or more guides and the second extension panel includes one or more guide fasteners slidably coupled to the one or more guides.

14. The attachment panel of claim 11, wherein the second extension panel includes a guide fastener operating as a cam latch that, when a lever of the guide fastener is depressed towards the first extension panel, the second extension panel is secured to avoid movement of the second extension panel in relation to the first extension panel.

15. A posterior collar assembly for a cervical collar, comprising:
a cervical collar posterior panel that comprises one or more cervical panels including a first cervical panel and a second cervical panel; and
an attachment panel including (i) a central support member coupled to the cervical collar posterior panel, (ii) a first extension member coupled to the central support member and including a first extension panel with at least one guide channel and a second extension panel with at least one guide fastener slidably coupled to the at least one guide channel, and (iii) a second extension member coupled to the central support member and configured to be extended laterally toward and away from the central support member.

16. The posterior collar assembly of claim 15, wherein the second extension member comprises a third extension panel and a fourth extension panel, the third extension panel being coupled to the central support member and including at least one guide channel and the fourth extension panel includes at least a guide fasteners that is slidably coupled to the at least one guide channel so that the third extension panel is able to laterally move away from or towards the central support member.

17. The posterior collar assembly of claim 1 being coupled to an anterior collar assembly to form a cervical collar.
